(19) United States
(12) Patent Application Publication
(10) Pub. No.: US 2025/0082500 A1
(43) Pub. Date: Mar. 13, 2025

(54) INTRAOCULAR DEVICES AND METHODS

(71) Applicant: Anant Sharma, Bedfordshire (GB)

(72) Inventor: Anant Sharma, Bedfordshire (GB)

(21) Appl. No.: 18/959,578

(22) Filed: Nov. 25, 2024

Related U.S. Application Data
(63) Continuation-in-part of application No. 18/829,652, filed on Sep. 10, 2024.
Application No. 18/829,652 is a continuation of application No. 16/499,437, filed on Sep. 30, 2019, now Pat. No. 12,083,041.

(30) Foreign Application Priority Data
Mar. 31, 2017 (GB) 1705177.2

(51) Int. Cl. A61F 9/00; A61F 9/007

(52) U.S. Cl. CPC A61F 9/0017; A61F 9/00745; A61F 9/00781; A61B 2217/005; A61B 2217/007

(57) ABSTRACT

The present application relates to novel intraocular devices and their use in surgical techniques, as well as the novel surgical methodology achieved from their use.

### INTRODUCTION

The present application relates to novel intraocular devices and their uses in surgical techniques, as well as the surgical methods that result from their use.

### BACKGROUND OF THE INVENTION

A cataract is a clouding of the lens inside the eye, causing vision loss that cannot be corrected without surgery. Cataracts are predominantly associated with the natural aging process and due to the increasing aged population globally, the prevalence of cataracts is expected to continue to grow significantly. Over 300,000 cataract operations are done per year in the UK alone. It is therefore desirable to provide safe, effective and efficient means for removal of the clouded lens (and replacement with an artificial intraocular lens IOL) to restore clear vision to patients.

De-risking the steps in cataract removal and techniques performed in other delicate eye surgeries are crucial to advancing this surgical field. Improvements, in cataract surgery particularly, are both technically challenging and commercially valuable; some severe complications, such as central visual loss following cataract surgery are not yet fully evaluated. Thus, understanding and controlling those factors which play a part in complications of this type of surgery remain of considerable interest.

Typically, a modern cataract removal procedure utilises a high-frequency ultrasound device that breaks up the cloudy lens into pieces; these are then gently removed from the eye by suction. Such a procedure is called phacoemulsification and can be performed with only relatively small incisions, which is beneficial for healing.

Most phacoemulsification involves a main incision of 2.0 mm-2.4 mm with either one or two smaller incisions for second instrument such as irrigation and aspiration apparatus. Another particular technique, known as bimanual micro-incision phacoemulsification, involves cataract extraction via two incisions of less than 1.5 mm diameter. Surgeons in the field consider that intraocular lenses capable of insertion through such dimensions will soon become widely available and hence this technique will grow in popularity; from a surgeon's point of view, such a surgical step is more advanced, offering improved anterior chamber stability and access to the capsular bag.

Hydrodissection is an essential step during the phacoemulsification procedure because it allows separation of the cortex from the capsule of the eye. The spatial separation facilitates easier and more complete cortical removal with less stress on the zonules; the rings of fibrous strands that connect the ciliary body with the crystalline lens of the eye. Hydrodissection also allows nuclear mobility to aid fragmentation of the cataract.

However, this step is still regarded by experienced ophthalmic surgeons as remaining an uncontrolled manoeuvre because during the hydrodissection stage particularly, it has been noted that the intraocular pressure (IOP) is often raised to a relatively high level (Khng C, Packer M, Fine IH, Hoffman RS, Moreira FB. Intraocular pressure during phacoemulsification. J Cataract Refract Surg. 2006; 32(2):301-8.). This situation may be exacerbated because the cannula being used to conduct the surgery in micro-incision phacoemulsification occupies a small diameter as compared to the incision wound and fluid readily moves out of the surgical wound in an uncontrolled manner. On some occasions, the iris of the eye will be pulled through the incision, causing an iris prolapse.

This surgical step therefore gives much cause for concern to surgeons as it routinely involves an uncontrolled risk factor resulting in one or more surgical complications. (http://webeye.ophth.uiowa.edu/eyeforum/tutorials/iris-prolapse-history.htm (accessed September 2017)).

Of particular concern is eye structure prolapse, particularly iris prolapse. This type of trauma is reported between 0.5% and 0.6% of all cataract surgeries (Ng DT, Rowe NA, Francis IC, et al. Intraoperative complications of 1000 phacoemulsification procedures: a prospective study. J Cataract Refract Surg. 1998; 24(10):1390-5.). Minor prolapses are often not reported.

Complications, such as iris prolapse, are more likely to occur in small pupils, Intra operative floppy iris syndrome (IFIS), shallow anterior chambers and small eyes. In particular diabetes, age, drugs, such as pilocarpine, previous surgery and inflammation are some of the contributing factors in a small pupil. It is further known that patients using certain drugs, for example, Tamsulosin, to treat prostate hypertrophy may also contribute to IFIS.

Techniques to reduce complications in these cases have been suggested in the art. However, they typically result in additional operative difficulty/risk and/or may not be suitable for certain groups of patient. For example, longer wound tunnels (incisions) make operative visualization challenging for the surgeon and risk increased astigmatism. Such long tunnel incisions may also not be possible in patients with previous corneal surgery or pathology.

It has been difficult for surgeons to identify/utilise suitable instruments during phacoemulsification that overcome this problem. Currently eye surgery is carried out in closed systems with a pump, e.g. peristaltic pump, which controls the exit and entry of fluid into the wound site. There can be insufficient irrigation in-flow which then further reduces anterior chamber stability. Such systems maintain an inflated eye shape to allow a workable surgical site during the procedure (to achieve this sometimes the bottle height is elevated but such action likely contributes to a raised IOP). Previously, surgeons have attempted to use pressurized irrigation to maximize inflow. However, increased inflow pressure also carries risk for elevated IOP if outflow from the eye ceases, even transiently.

Rises in IOP, even after the hydrodissection step, continue to present a problem for some patients and therefore mitigating such a risk is challenging for surgeons. IOP has been shown to rise significantly throughout cataract surgery (Yune Zhao, Xingyu Li, Aizhu Tao, Jianhua Wang, and Fan Lu Intraocular Pressure and Calculated Diastolic Ocular Perfusion Pressure during Three Simulated Steps of Phacoemulsification In Vivo. IOVS, June 2009, Vol. 50, No. 6. Pages 2927-31) and it can cause damage to the eye, especially in patients with glaucomatous optic nerve damage, a condition which is more sensitive to IOP rise. For example, insertion of the intraocular lens toward the end of the cataract surgery is also a surgical step which significantly raises the IOP.

Finally, IOP rise can also occur after cataract and other eye operations such as glaucoma filtration procedures (trabeculectomy, tube drainage surgery), corneal transplant surgery, cellular transplant surgery, retinal and/or vitreous surgery. This is known to damage the optic nerve with wipe-out of the visual field and central vision and other complications such as permanently dilated pupil, corneal oedema (swelling) with visual loss. In high risk cases medication such as diamox tablets or eye drops is given to reduce IOP, however the IOP can still rise (Kass MA, Gordon MO, Gao F et al. (2010). Delaying Treatment of Ocular Hypertension: The Ocular Hypertension Treatment Study. Arch Ophthalmol 128 (3), 276-287).

IOP has also been known to rise post-surgery in normal patients and cause damage such as reduced vision, dilated pupil and loss of visual field. IOP rises after cataract surgery is remains an important and common problem since IOP rise can exacerbate vision loss particularly in glaucoma patients who have pre-existing visual field defects (Tranos et al., 2014).

It is therefore considered that in the field of ocular surgery there is a need for improvements in techniques and apparatus to manage or reduce the risk associated with uncontrolled steps of that surgery.

In particular, in modern day cataract surgery there is a clear need for new procedures, techniques and surgical tools to reduce the risk of operative and post-operative complications. This is especially true as it concerns modern phacoemulsification to prevent iris prolapse and other significant complications associated with a rise in IOP, whether occurring during surgery or post operatively.

The present invention arises from a desire by the applicant to address the technical issues associated with the risks of ocular surgery, as described above.

### SUMMARY OF THE INVENTION

The present invention concerns a device for use in ocular surgery and/or post-operative surgical care comprising a solid core structure adapted to form a peripheral seal with an ocular layer(s) about the device in-situ, the structure comprising at least one internal channel adapted to balance intraocular pressure by permitting at least one of a fluid or gas to pass through the structure.

Such a device may be safely placed and/or secured past the pupil margin, for example, during cataract surgery. However, for other types of surgery, the device may be placed elsewhere in the eye, including the cornea, limbus, sclera, suprachoroidal space, or in the layers of the conjunctiva, such as subconjunctival or subtenon.

Techniques/manoeuvres such as hydro-dissection, as described during cataract removal, can be carried then out by the surgeon with reduced risk of complication.

For example, after placing the device and initiating hydrodissection, any excess fluid in the eye may safely move from an internal chamber, at the working surgical site of the cataract, towards a distal end of the device and will flow via the internal channel of the core structure and out the proximal end, which is external of the corneal outer layer. Essentially, the presence of the device in the eye therefore prevents the build-up of unwanted IOP. Further, the device enables the pressure to be balanced or a build-up to be passively neutralised without requiring any external pumping action.

In particular, the device of the invention allows the surgeon to continue to undertake a surgical procedure in the eye with reduced concern about uncontrolled change in IOP. Further, this removes the need to take urgent steps to rectify, if possible, complications that result, such as an enhanced risk that essential eye structures, such as the iris are dragged out of the eye.

The solid core may be adapted to form such a seal by, for example, being compressible, deformable or otherwise malleable to ensure the device can be inserted easily through an incision but remain fitted to the ocular layer(s) through which it passes. In preferred embodiments the core structure of the device may be circular or ellipsoid in cross-section.

The core structure of the device typically comprises a proximal end (proximal to the user/surgeon) and a distal end.

In embodiments, the device may range from 0.01 mm to 50 mm in length, that is, the measurement between the two ends of the device. In some embodiments the structure is elongate and hence shaped like a tube or sleeve-like.

The core structure of the device may further comprise a retaining feature to help prevent the device from becoming displaced. The retaining feature may be a corrugated outer surface for enhanced grip with the ocular layer(s). In other embodiments the elongate structure may have a screw thread configuration to allow to the device to be more securely fixed in the eye.

In further embodiments, the core structure may additionally or alternatively have a retaining structure with an outer surface comprising angled protrusions, in the form of a plurality of feet, flaps or wings, evenly spaced in a circumferential manner or a single angled annular flap which may permit the device to be inserted though the cornea, or other structure of the eye, and which may temporarily retain the device in the inserted position thereafter by resting against an inner domed surface of that structure, such as the cornea.

Typically, the device of the invention may have an average width or cross section ranging from 0.001 mm to 15 mm in size.

The diameter (or size of cross section) at the proximal end may be the same or different to the distal end. In some embodiments the cross section of the structure is constant.

In other embodiments the cross section of the structure may vary, or have a sliding gradient. For example, the device may taper toward the distal end, whereby the cross section of the distal end is narrow as compared to the cross section at the proximal end. In such embodiments the tapered end permits ease of entry when inserting the device into the eye. Such a configuration may allow the surgeon to make the incision with the device itself, hence dispensing for the need of an additionally pre-made incision with a different tool. The edge of the distal end of the device may be further shaped to facilitate penetration through the layer(s) of the eye. In some cases the distal end maybe round-ended with side ports, in other examples the distal end may be be bevelled, chiselled or sharp to enhance the ease of placement.

In some embodiments the structure of the device comprises walls which range from 0.0001 mm to 5 mm in thickness.

The device may preferably be formed from an elasticated material to enable the surgeon to easily place the device within a very small incision in the eye and therefore permit the peripheral seal to be tight with the ocular layer(s). The device may be formed from a material biocompatible with ocular tissue such as plastics or silicone, including grade a silicone. Such materials include but are not limited to medical grade silicone, silicone polymer, silicone rubber, rubber, latex, Teflon, polypropylene, nylon, plastic and thermoplastic polyurethanes.

In some embodiments the structure of the device comprises a plurality of internal channels. Said multiplicity of channels can serve to provide optimal irrigation and balancing of IOP functioning to promote effective drainage. The one or more further internal channels may provide different functionality, so the device is adapted to permit efficient release of gas or fluids of differing viscosity, in addition to the removal of excess physiological fluid, such as balanced salt solution, isotonic saline or viscoelastic fluid such as sodium hyaluronate, hypromellose.

The device may further comprise further features to enhance or promote movement of physiological or non-physiological fluid out of the eye when IOP is raised. In some embodiments the movement is promoted by a feature positioned within the internal channel, such as grooves or valves. In some embodiments, the movement of the fluid is able to be controlled by one or more of those valves, for example by one-way valves. In other embodiments the valve are pressure valves. In embodiments the pressure values are pre-set such that when pressure exceeds the setting the valve will open to allow the fluid to escape the proximal end of the structure of the device. Such pressure valves allow a closed system to be retained as far as possible without risking uncontrolled rises in IOP. Such a pressure valve may be positioned within the internal channel of the core structure.

In some further examples of the invention, the structure of the device may comprise an annular flange at the proximal end of the core structure which may sit on the surface of the eye structure, such as the cornea. This feature allows the maximum depth of the device during insertion into the eye structure to be pre-determined and thus controlled when the length of the core structure is known. It prevents the device from being embedded too deeply in the eye and also facilitates insertion and removal.

The device of the invention may be used in situ, temporarily, during surgery and removed once the surgical procedure is complete. In such embodiments the device is therefore removable and may comprise features described herein to further facilitate that removal.

However, the device may instead form a permanent seal with the layers of the eye and thus either remain embedded therein permanently, or until it naturally dissolves. In such embodiments, the device may be made from a biodegradable or non-biodegradable material.

In examples relating to post-surgical or non-surgical associated treatment of IOP, where it is intended to install the device for post-surgical care, or leave the device in position thereafter, the device may be made of a dissolvable material. The material of the device, once in situ, is biodegradable and dissolves to close the wound within 6 weeks of insertion. Such an embodiment helps ensure safe wound closure whilst continuing to keep the IOP balanced for a minimum period of time post surgery. This is especially useful where IOP continues to be a high risk factor post-surgery, for example in certain patient groups e.g. glaucoma. Alternatively, the non-biodegradable embodiment of the device can simply be removed after a minimum period of time.

There is further disclosed, a kit comprising a plurality of ocular devices according to any of the before described embodiments, wherein at least two of the devices accord to different embodiments of the invention as herein before described. It is considered that the invention may be provided as a kit to the surgeon so he/she may select the appropriate device according to the surgical need. For example, a patient with a particular risk or with smaller eyes may require a differently sized device. It is therefore valuable that the surgeon has a choice of appropriate devices from which he may choose each having the essential feature of the invention.

Furthermore, in another aspect the invention concerns a surgical apparatus including for example a hydrodissection device or a lens injection device, for use in ocular surgery comprising: an elongate cannula shaft for the introduction of fluid or gas into an ocular space and a hollow annular, flexible, sleeve surround, wherein the sleeve comprises a plurality of apertures to permit excess fluid or liquid in the ocular space to re-enter the device to exit the ocular space thereby balancing intraocular pressure during surgery.

In this aspect of the invention the device provides both the function of introducing the fluid necessary for hydrodissection and still solves the technical problem but does so in a slightly different way. Here the excess fluid, whether physiological fluid and/or other fluids needed in the procedure such as viscoelastic solution used during introduction of the new lens into the eye.

The invention of the applicant extends to a basic solid (preferably compressible or flexible) device in accordance with the many embodiments herein before described may be utilised with a normal hydrodissection cannula or lens injector. For example, in such embodiments, the device forms the “sleeve surround” and necessarily includes a series of apertures provided therein. In such embodiments the device is therefore temporary or removable and may be selected to retro-fit an existing cannula to create a hydrodissection device for use during surgery. The removable device may be disposable after use with the cannula in this form or in some embodiments it may be possible detach the cannula from the device but leave the basic device in the eye thereafter for post-surgical maintenance.

In another aspect the invention relates to use of the device or the surgical apparatus incorporating the device of the invention, or any embodiment thereof, in ocular surgery. In a further aspect the invention relates to use of the device or the surgical apparatus in cataract surgery. In another aspect the invention relates to use of the device or surgical apparatus, in any embodiment described, in phacoemulsification surgery.

In a further aspect the invention relates to use of the device, or the surgical apparatus incorporating the device as described herein, in a hydro-dissection or other step of phacoemulsification surgery, angle surgery such as schlemm canal or trabecular surgery including stents, implants or vitreoretinal surgery.

In another aspect the invention concerns use of device for post-surgical maintenance of intraocular pressure in the range of 3<30 mmHg.

In another aspect the invention relates to a method of controlling intraocular pressure during surgery to the eye comprising inserting the device or surgical apparatus incorporating the device according to any embodiment of the invention.

In a further aspect the invention concerns a method of maintaining an intraocular pressure in the range of 3 to <30 mmHg during surgery to the eye comprising inserting the device or surgical apparatus according to any embodiments described.

In some aspects or embodiments, a device is provided for temporary use in cataract surgery and post-operative surgical care is described which includes an elongate tubular structure defining at least one internal channel therein. The internal channel extends between a proximal end of the tubular structure and a distal end of the tubular structure. The proximal end of the tubular structure includes an annular flange extending axially therefrom with a vertical wall extending proximally from the annular flange. The annular flange and the vertical wall defining a bore therein which is configured to receive (i.e., maintain, secure, stabilize) a fluid injection device therein. The bore is in fluid communication with the internal channel such that fluids exiting a patient to balance intraocular pressure are capable and/or configured to pass through the internal channel to the bore. The bore includes one or more grooves defined along the annular flange and/or the vertical wall. The one or more grooves are also in fluid communication with the internal channel and/or the bore such that fluids exiting a patient to balance intraocular pressure are capable and/or configured to pass through the internal channel to the one or more grooves to exit the device.

In particular, the present invention further concerns a method of surgical cataract removal comprising the steps of: viscoelastic insertion, capsulorhexis, hydrodissection; intraocular lens insertion; phacoemulsification; irrigation; lens aspiration and viscoelastic fluid removal, wherein during one or more of the above steps the device or surgical apparatus, as previously described, is used to balance intraocular pressure throughout the removal.

An uncontrolled change in IOP, described in the before-mentioned background to the invention is associated with a number of complications and risk factors during surgery to the eye. Therefore, maintaining a safe IOP during any eye surgery technique or step in which IOP is characteristically a risk factor is beneficial to the patient and to the surgeon since it prevents an uncontrolled factor effecting the flow, efficiency and precision of the procedure as a whole. The benefit of the invention however has been found particularly beneficial in cataract removal and specifically procedures that require hydrodissection.

### BRIEF DESCRIPTION

Various features, embodiments and examples of the presently disclosed invention including the device, methods will now be described herein with reference to the accompanying figures wherein:

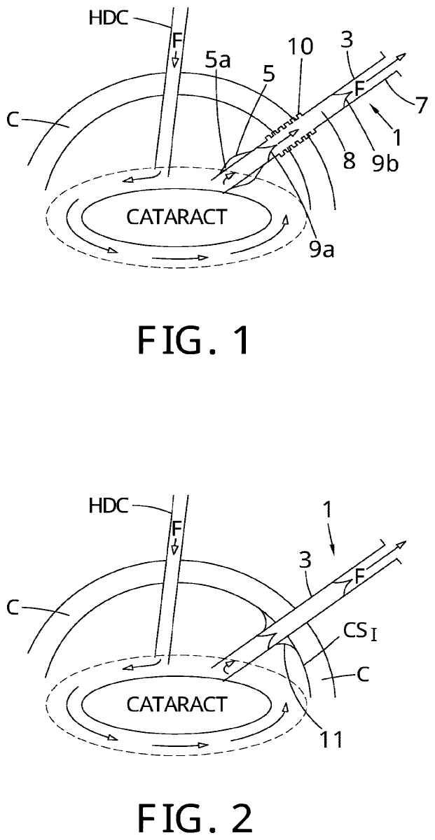

FIG. 1 illustrates a first aspect of the device of invention positioned relative to the structure of the eye;

FIG. 2 illustrates an embodiment of the invention;

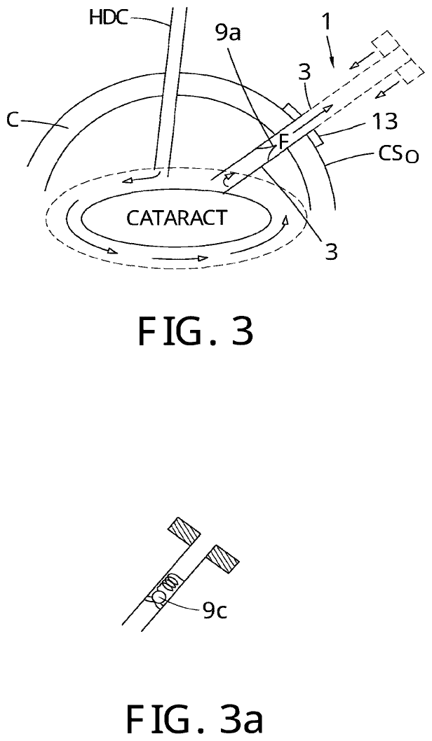

FIG. 3 illustrates a further embodiment of the device of the invention;

FIG. 3a illustrates a number of further embodiments of the device;

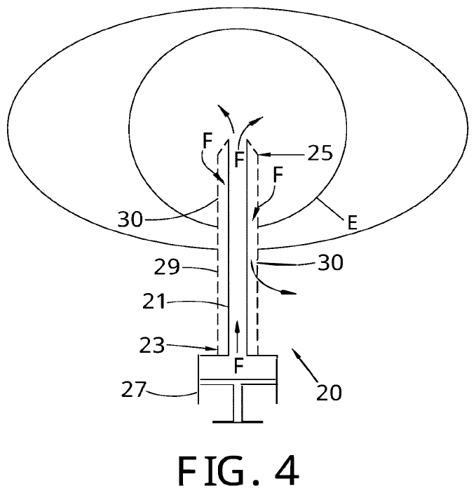

FIG. 4 illustrates a further aspect of the invention in which a surgical apparatus such as a hydrodissector incorporates a device;

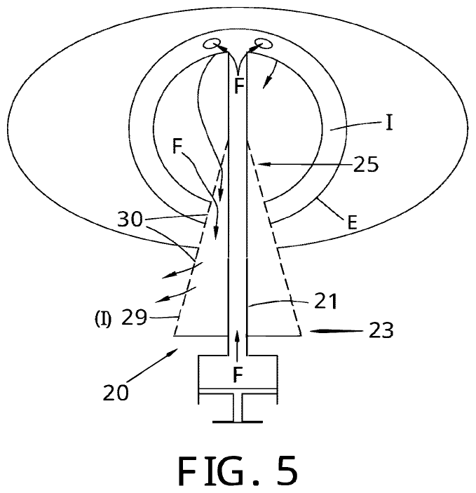

FIG. 5 illustrates embodiments in which device of the invention when used in combination with an instrument of surgery is either removable or integrated with an instrument used in ocular surgery;

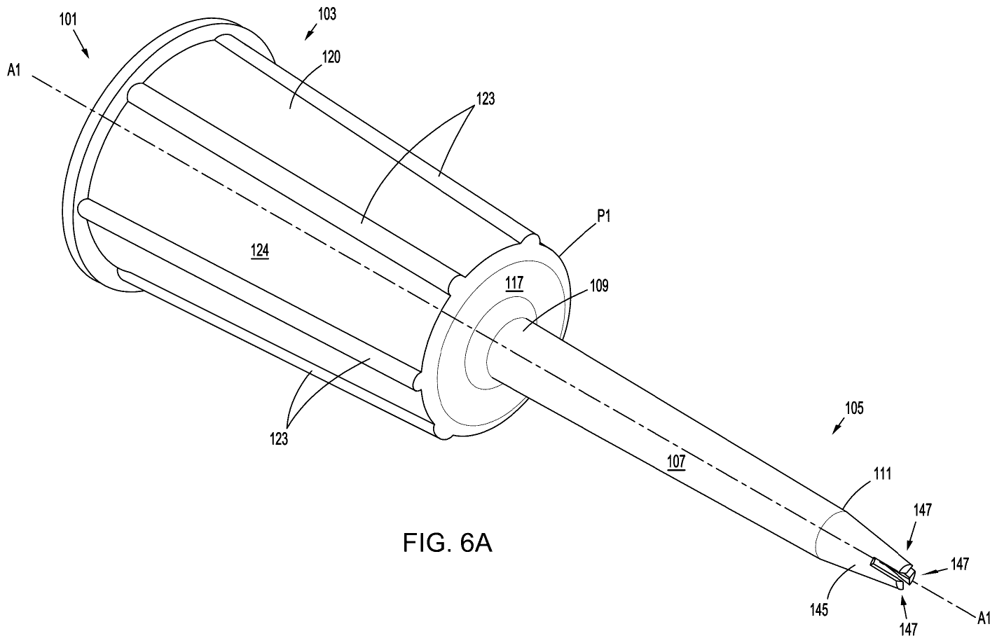

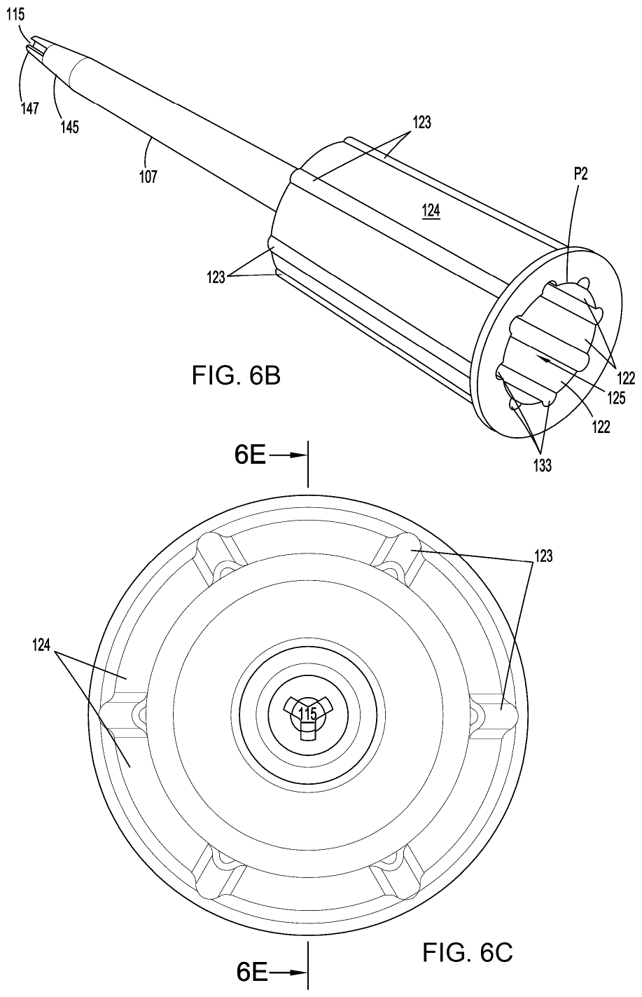

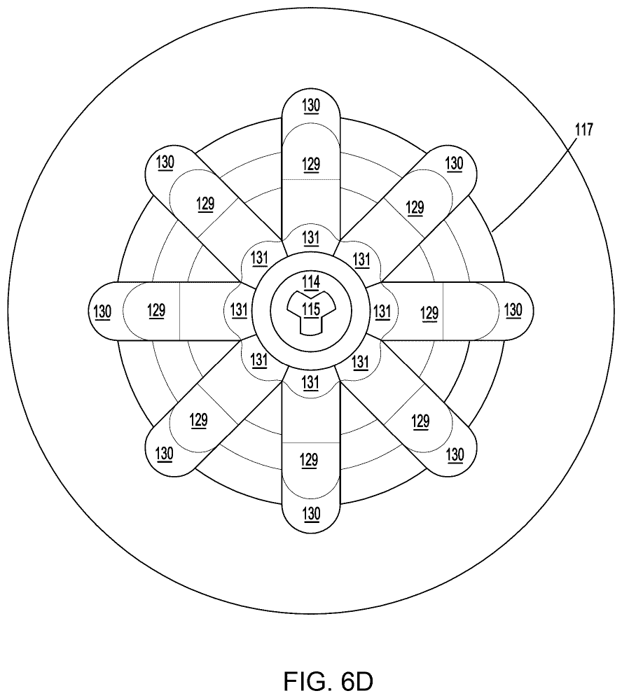

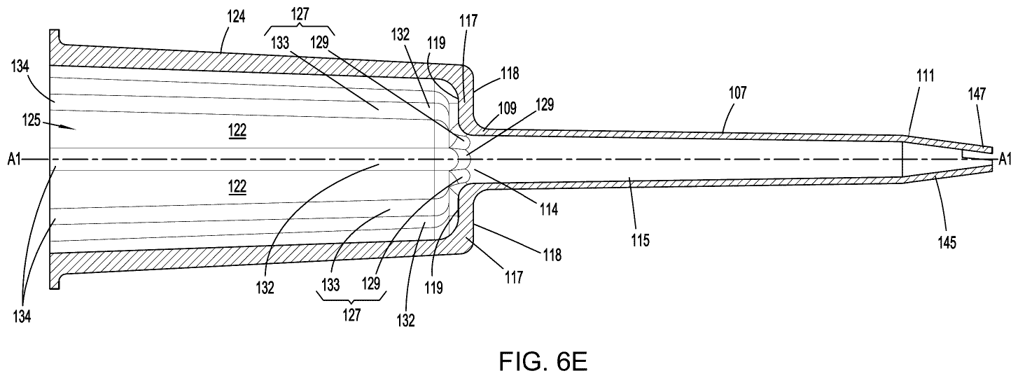

FIGS. 6A and 6B are a front and rear perspective view, respectively, of an intraocular device as provided in one or more embodiments herein;

FIG. 6C is a bottom view of the intraocular device of FIGS. 6A and 6B as provided in one or more embodiments herein;

FIG. 6D is a top or proximal view of an annular flange of the intraocular device of FIGS. 6A and 6B as provided in one or more embodiments herein;

FIG. 6E is a cross-sectional side view of the intraocular device of FIGS. 6A and 6B as provided in one or more embodiments herein;

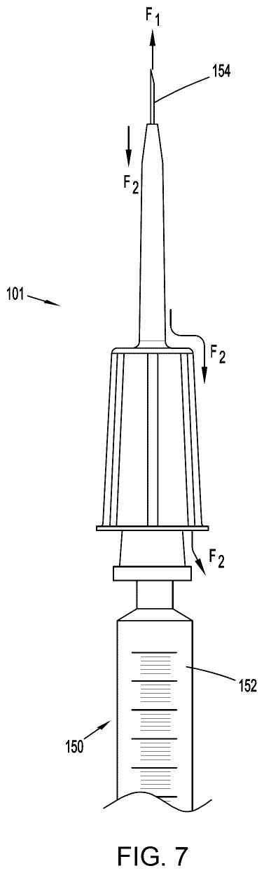

FIG. 7 is a side view of the intraocular device of FIGS. 6A and 6B in combination with a fluid injection device as provided in one or more embodiments herein;

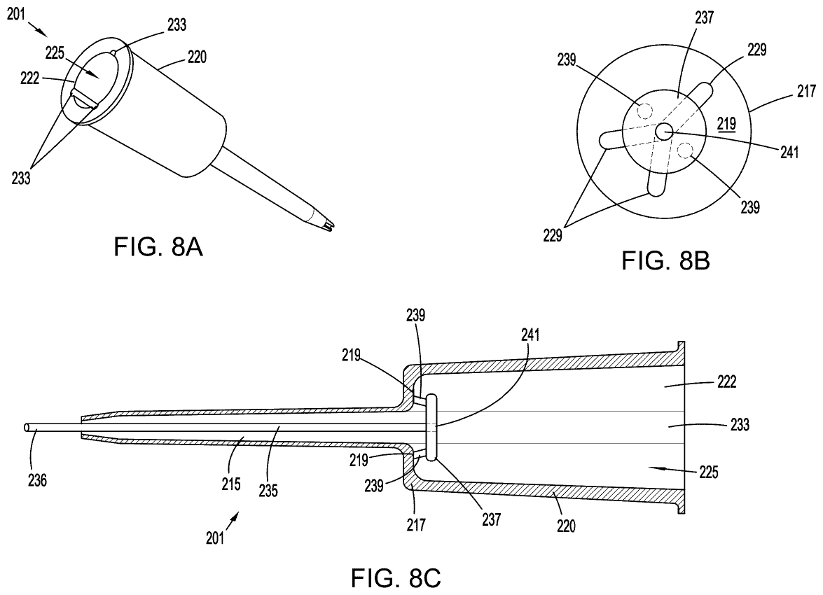

FIG. 8A is a front perspective view of an intraocular device as provided in one or more embodiments herein;

FIG. 8B is a top or proximal view of an annular flange of the intraocular device of FIG. 8A as provided in one or more embodiments herein;

FIG. 8C is a cross-sectional side view of the intraocular device of FIG. 8A as provided in one or more embodiments herein;

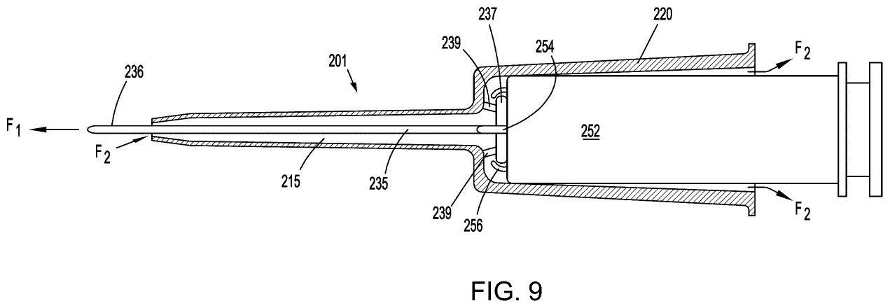

FIG. 9 is a cross-sectional side view of the intraocular device of FIG. 8A in combination with a fluid injection device as provided in one or more embodiments herein;

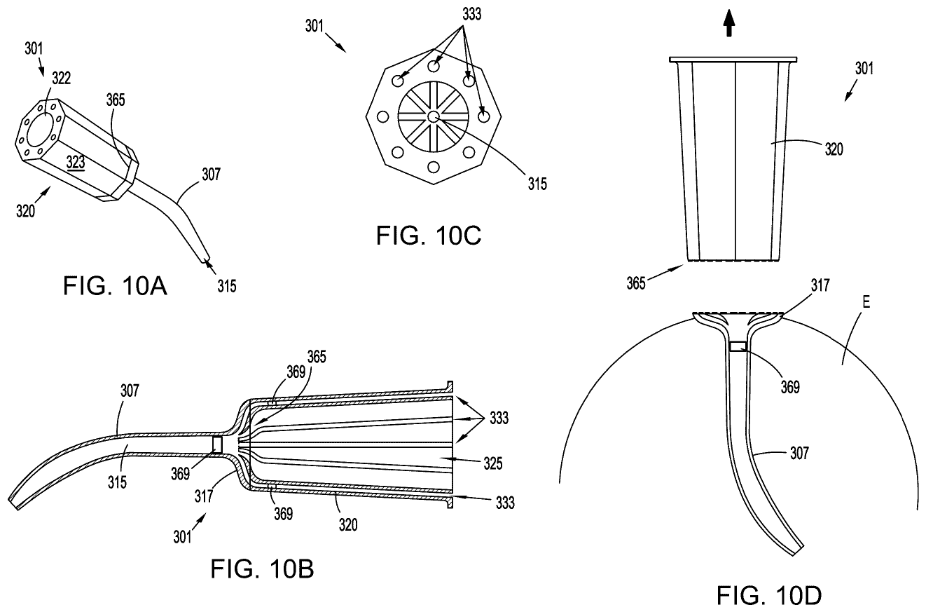

FIG. 10A is a front perspective view of an intraocular device as provided in one or more embodiments herein;

FIG. 10B is a cross-sectional side view of the intraocular device of FIG. 10A as provided in one or more embodiments herein;

FIG. 10C is a top or proximal view of the intraocular device of FIG. 10A as provided in one or more embodiments herein; and

FIG. 10D is a schematic representation of the intraocular device of FIG. 10A temporarily implanted in an eyeball as provided in one or more embodiments herein.

### DETAILED DESCRIPTION

As used herein, the term “proximal” refers to the end of the apparatus or feature which is closer to the user or clinician and the term “distal” refers to the end of the apparatus which is relatively speaking distanced from the user as compared to the proximal end.

Variations of the presently disclosed device are possible and within the scope of the present disclosure provided the major features of the invention are present, as defined by the claims.

In FIG. 1 the device of the invention is shown in-vivo as would be seen according to a rough cross-section of the structure of the eye. In this embodiment the device is positioned during ocular surgery, in particular during a cataract removal procedure, for example. However, the device in any of the examples described, wherein it is not permanently part of a larger surgical device such as a hydro-dissector or lens injector, could equally remain in place post-operatively. Alternatively, the device shown could be inserted purely for IOP care or maintenance, in order to obtain the effect, without having been directly used during formal surgery, per se.

During hydrodissection step of cataract removal, the surgeon must insert a hydrodissection cannula HDC through the cornea, as shown in the figure. After capsulorhexis, the device 1 of the invention will be inserted at different location.

The device 1 has a deformable core, or central structure, shown as section 3. When inserted in to the eye, the core compresses and its external surface adapts with the ocular structure surface such as the cornea C, through which it is passed, forming a peripheral seal therewith. The core structure has a distal end 5 and a proximal end 7, with an internal channel 8 extending there through. The device is a compressible or deformable material to enable a very small incision in the eye and yet permit the peripheral seal to be tight. The device may be formed from one or more functional material biocompatible with ocular tissue such as flexible plastic or silicone. In preferred examples, such as this, the device is formed from any material, or materials in combination, conforming to European and International regulatory requirements for surgical devices and implants.

The surgeon may make a micro incision (currently 0.5-3 mm in cataract surgery) in order to apply the device to the position shown in FIG. 1. However, the device may have features to allow for self-insertion without the need for a pre-incision. For example, in some examples or embodiments the distal end 5 may be bevelled or pointed such that the device facilitates self-insertion. In some examples, the distal end 5 may alternatively or additionally be made of a slightly harder material Sa to enable insertion with the device alone.

In the example shown, the core structure 3 with a substantially circular cross-section and the device is generally elongate with a tube or sleeve-like shape. Importantly, the selection of a particular measurement combination may be made such that the device is suitable in view of a
patient's eye shape and dimension. The width or cross-section dimension of the device is typically in the range of 0.001 mm to 15 mm in size, whereas the length may range from 0.01 mm to 50 mm. The surgeon may therefore select an embodiment of the device, corresponding to a combination of the ranges, herein described, to provide a suitable patient match, especially if the device is intended to remain in the patient's eye post-surgery or inserted independently into the eye. In this regard, an embodiment of the invention concerns a provision of a device kit in which a selection of the most common combination is provided for in one useful pack.

In the example shown, the diameter of the core structure is generally constant. In this example, the core structure also comprises a corrugated section 10 (but it may be corrugated throughout). The corrugated outer surface of the structure of the device enhances grip with the corneal layer(s) and after insertion helps prevent the device from becoming displaced (during the rest of the procedure) since the structure (due to the internal pressures) may be liable to slip from the original position in a proximal direction.

Once the device is safely placed and/or secured past the pupil margin hydro-dissection may proceed. On initiating hydrodissection, any excess fluid introduced into the eye by the in the eye may safely move from the internally closed chamber at the working surgical site of the cataract towards the distal end of the device and flows (shown by the directional arrows and F) via the internal channel out the proximal end. The device therefore prevents the build-up of unwanted IOP and the full procedure of cataract removal can be carried out with reduced risk of complication that is associated with unbalanced IOP. Furthermore, since the device provides a secure seal, the eye remains expanded and does not collapse even once excess fluid has escaped from the surgical site.

As shown herein, the device enhances or promotes movement of physiological fluid out of the eye when IOP is raised. This movement is further promoted by at least one valve 9 positioned within the internal channel of the core structure; here two valves 9a, 9b are shown to regulate the movement of fluid more precisely. Such valves include one-way valves, for example. In particular, the movement of the fluid is able to be controlled and the IOP balanced even more precisely when multiple valves within the internal channel 8, in this example two, are positioned either side of the corneal wall.

A further embodiment of the device is provided in FIG. 2. Here the device of the invention is similarly constructed in its main elements as before described in FIG. 1. However, the retaining structure which secures the core structure in this example is a stabiliser 11 comprising an outer surface angled protrusion or protrusions, for example in the form of a plurality of feet, flaps or wings evenly spaced in a circumferential manner or a single angled annular flap. The stabiliser permits the device to be inserted though the cornea in the manner previously described but will act to temporarily retain the device in the inserted position specifically by resting against an inner concaved surface CSI of the cornea incision layer. The device may be retained against other layers of the eye, e.g. sclera, choroid, parsplana.

During glaucoma or combined glaucoma/cataract surgery, the device therefore acts as IOP balance mechanism but also, crucially, postoperatively functions to allow the eye to inflate if over drainage occurs.

FIG. 3 provides a further embodiment in which different types of valve may be used together to provide enhanced control of fluid movement and ease of use. In this embodiment, the device of the invention comprises an annular flange or lip 13 at the proximal end of the core structure which may sit of the surface of the cornea once the device is inserted. During insertion this feature stops the device (at the proximal end) from penetrating the cornea past beyond a maximum depth (which is pre-determined as the length of the core structure in relation to the flange is known) and lies upon an outer convex surface of the cornea CSO. The flange therefore facilitates both insertion and removal of the device. This configuration helps control and distance the device, when in situ, from the posterior surface of the cornea, especially in eyes with shallow anterior chambers thereby avoiding potential injury. In an additional embodiment the flange maybe adjustable along and temporarily fixed in place on the compressible structure 3 such the proximal end of the device protrudes therefrom (shown in dash).

The device in another embodiment, shown in FIG. 3a, may instead comprise an internal valve, in this case a spring-based valve 9c, which can be pre-set to function under particular pressure ranges. However, other valves such as more simplistic flap valves and one-way valves, such as 9a, may equally be utilised in this combination. Alternatively, such valves can be electronic and/or magnetic and control valve pressure such that they may be adjusted when device is in situ.

In addition to that configuration, in other examples, the flange may also function as a pressure valve by temporarily sealing the proximal end of the device. The flange is adapted to deform under pressure from fluid in the internal channel to create a temporary aperture at the proximal end of the device when the IOP rises and fluid moves into the internal channel.

In any case, a pressure value can pre-set such that when pressure exceeds the setting in the internal channel the valve will open to allow the fluid to escape the proximal end of the structure of the device. In particular, pre-set pressure valves allow a closed system to be retained as far as possible without risking uncontrolled rises in IOP and only one valve need be utilised for this function although they may be used in combination.

FIG. 4 shows a hydrodissection device 20 with proximal and distal ends, 23 and 25 in accordance with a further aspect of the present invention. Such a device comprises a central shaft 21. The device typically comprises a fluid injection means or apparatus 27 at the proximal end 23 of the device for inserting fluid into the ocular space during surgery. The device further includes a hollow annular flexible sleeve surround 29, wherein the sleeve comprises a plurality of apertures 30 spaced there along to permit excess fluid or liquid in the ocular space to re-enter the device to exit the ocular space thereby balancing intraocular pressure during surgery. A simplistic illustration of the movement of fluid F is shown by arrows when the device is utilised in surgery of the eye E.

The invention of the applicant also covers the embodiment in which a basic compressible device in accordance with the many embodiments hereinbefore described as 1 may be utilised with a central shaft hydrodissector, for example, as the “flexible sleeve surround” 29 when the compressible device 1 necessarily includes a series of apertures therein (shown as 30 in the present embodiment). In such embodiments the sleeve/basic device may be selected to retro-fit the desired cannula to create a hydrodissection device. The sleeve may therefore be disposable after use with the cannula in this form. The hydrodissection sleeve cannula can be attached/detached to the syringe.

The device in combination therefore usefully provides both the function of introducing the fluid necessary for hydrodissection step of surgery and still solves the technical problem relating to IOP build up, but does so in a slightly different way since such a device when used with a cannula necessarily requires apertures along, whereas when used alone this feature must not be included.

In other embodiments, as shown in FIG. 5, the removable/adjustable compressible device 1 is either used with an instrument such as a cannula, or wherein the sleeve 29 is integrated with the cannula of a hydrodissection device for example. In such embodiments, the cross-section of the sleeve surround has a sliding gradient; the device 1/sleeve 29 taper towards the distal end, whereby the cross section of the distal end 25 is narrow as compared to the cross section at the proximal end 23. In such embodiments the tapered end permits ease of entry when inserting the device into the eye. Such a configuration may in particular allow the surgeon to make a micro incision with the device itself, hence dispensing for the need of an additionally pre-made incision with a different tool. The edge of the distal end of the device may further be bevelled, chiselled or sharp to enhance the ease of placement. By virtue of the tapering of the sleeve, a peripheral seal with the eye is formed allowing for an improved device.

During use of the surgical apparatus, the fluid F flows down the shaft 21 of the cannula and into the eye, where after, excess fluid F is enabled to return from under the iris I back into the device via the apertures 30 in the removable device 1 or sleeve 29 thereby escaping the ocular space, avoiding IOP from rising.

In other embodiments the excess fluid/gas may include more than one physiological/non-physiological fluid or gas. For example, viscoelastic fluid is needed in the procedure during introduction of a new lens into the eye. A lens injector filled with such fluid and a lens and injected in a closed system necessarily raises the pressure in the eye further. However, the invention is equally useful here; this additional fluid may also be safely removed by the same mechanism. In particular, the device can be placed temporarily through paracentesis when lens is being inserted to allow viscoelastic and/or other liquid already present in the ocular space to escape safely via the device 1 or sleeve 29, rather than increase the IOP beyond 30 mmHg, thereby potentially damaging intraocular structures, such as the zonules or capsule.

FIGS. 6A-6E depict various views of an intraocular device 101 as provided in yet another embodiment and/or aspect. The device 101 extends between a proximal end 103 including a conical or cylindrical portion and a distal end 105 including a narrower elongate tubular portion. The length of the conical or cylindrical portion may be generally equal to the length of elongate tubular portion, while the width of the conical or cylindrical portion may be greater than the width of the elongate tubular portion.

The device 101 is configured to be used temporarily in eye surgeries, such as cataract and/or glaucoma surgeries. The device 101, in whole or in part, may remain in the eye post-operatively for 1 day-6 weeks. In some embodiments, the device 101, in whole or in part, may remain in the eye post-operatively for 2 days-2 weeks. In some embodiments, the device 101, in whole or in part, may remain in the eye post-operatively for 3 days-1 week

The device 101 includes an elongate tubular structure 107 having at least one internal channel 115 extending between a proximal end 109 of the tubular structure 107 and a distal end 111 of the tubular structure 107. As depicted in at least FIGS. 6A, 6B, and 6E, in some embodiments, the elongate tubular structure 107 may be a generally linear elongate tubular structure 107. The linear tubular structure 107 may define a constant outer perimeter along a length thereof.

An annular flange 117 extends outwardly (and/or axially) from the proximal end 109 of the tubular structure 107. The annular flange 117 extends generally perpendicular to a longitudinal axis A₁ of the tubular structure 107. The annular flange 117 defines a thickness between a patient-facing (and/or distally-facing) first surface 118 and an opposite user-facing (and/or proximally facing) second surface 119. The distal end 109 of the tubular structure 107 includes a forked portion 145 having a plurality of tines 147 configured to penetrate eye tissue, with and/or without an incision. In some embodiments, the forked portion 145 includes 3 or more tines 147.

The device 101 further includes a vertical wall 120 positioned proximally to the tubular structure 107 and the annular flange 117. The vertical wall 120 extends in a proximal direction away from the annular flange 117. The vertical wall 120 wraps around the annular flange 117 at or near an outer perimeter p₁ of the annular flange 117. The vertical wall 120 and the annular flange 117 define a bore 125 therein. The bore 125 is in fluid communication with a proximal end 114 of the internal channel 115. The bore 125 is configured to receive a fluid injection apparatus (not shown in FIGS. 6A-6E) therein.

The vertical wall 120 defines a thickness between an inner surface 122 of the vertical wall 120 and an outer surface 124 of the vertical wall 120. The inner surface 122 of the vertical wall 120 generally defines an outer perimeter p₂ of the bore 125. The outer perimeter p₂ of the bore 125 may be sized to receive the fluid injection apparatus therein. The outer surface 124 may include one or more raised ridges 123 positioned thereon for handling of the device 101.

In some embodiments, as shown in at least FIGS. 6A, 6B, and 6E, the vertical wall 120 tapers distally toward the annular flange 117 to form a conical vertical wall 120 (and/or a conical bore 125).

The device 101 further includes one or more grooves 127 in fluid communication with the proximal end 114 of the internal channel 115 to permit physiologic fluids to pass through the device 101 to balance intraocular pressure before, during, and/or after an eye procedure. As shown in at least FIG. 6E, the one or more grooves 127 extend along and may be defined within the annular flange 117 and the vertical wall 120.

In some embodiments, as further shown in at least FIG. 6E, the one or more grooves 127 include a plurality of grooves 127 spaced radially about the annular flange 117 and the vertical wall 120. Each groove 127 of the plurality of grooves includes a flange groove part 129 in fluid communication with a wall groove part 133.

As shown in FIG. 6E, the flange groove part 129 is defined into the second surface 119 of the annular flange 117 to create a duct and/or pathway for physiological fluid to travel therein when exiting the eye. As shown in FIG. 6D, the flange groove part 129 may extend radially along the second surface 119 of the annular flange 117 between an inner flange groove part 131 (located at or near the internal channel 115) and an outer flange groove part 130 (located at or near vertical wall 120).

The flange groove part 129 is in fluid communication with: the proximal end 114 of the internal channel 115 via inner flange groove part 131; and, the wall groove part 133 via outer flange groove part 130. In some embodiments, the flange groove part 129 is an open flange groove which is open to and in fluid communication with a distal end of the bore 125.

As further depicted in FIG. 6D, the flange groove parts 129 may be arranged across the annular flange in a symmetrical pattern, such as the flower-petal design as shown. However, the flange groove parts 129 may be alternatively arranged in an asymmetrical manner (See FIG. 8B).

As further shown in FIG. 6E, the wall groove part 133 may be defined into the inner surface 122 of the vertical wall 120 to create a duct and/or pathway for physiological fluid to travel therein when exiting the eye. The wall groove part 133 extends vertically (and/or proximally from the flange 117) along the vertical wall 120. A distal end 132 of the wall groove part 133 is in fluid communication with a flange groove part 129, and particularly outer flange groove part 130.

In some embodiments, the wall groove part 133 is an open wall groove part 133 which is open to and in fluid communication with bore 125.

As further depicted in FIGS. 6B, the wall groove parts 133 may be arranged along the vertical wall 120 in a symmetrical pattern as shown. However, the wall groove parts 133 may be alternatively arranged in an asymmetrical manner (See FIG. 8A).

In some embodiments, as further depicted in FIG. 6B, the wall groove parts 133 are radially spaced around the outer perimeter p₂ of the bore 125 intermittently enlarging portions of the outer perimeter p₂ of the bore 125 to permit physiological fluid to pass through the open wall groove parts 133 while the fluid injection device is received within the bore 125. Particularly, exiting physiological fluids are allowed and/or forced to travel up along the wall groove part 133 against exterior parts of the fluid injection device positioned within the bore 125. The physiological fluid exits around an outside of the fluid injection device. The wall groove parts 133 and the vertical segments of the inner surface 122 of the vertical wall 123 alternate around the outer perimeter p₂ of the bore 125.

The outer perimeter p₁ of the annular flange and/or the outer perimeter p₂ of the bore 125 is greater than an outer perimeter p₃ of the elongate tubular portion 107.

In some embodiments, the length of the elongate tubular portion 113 is generally equal to the length of the vertical wall 120 in combination with the thickness of the annular flange 117.

FIG. 7 illustrates the device 101 combined with a fluid injection device 150, such as a syringe barrel 152 including a needle and/or cannula 154 extending distally therefrom. As illustrated, the needle and/or cannula 154 extends through and beyond elongate tubular portion 107 to deliver fluids F₁ into an eye (not shown), while a distal end of the injection device 150 (and/or syringe barrel 152) is secured inside vertical wall 120 (and/or bore 125) such that physiological fluids flow F₂ out of the eye (and/or the intraocular device 101) under pressure via the one or more grooves.

FIGS. 8A-8C depict various views of yet another intraocular device 201 as provided in yet another embodiment. Intraocular device 201 is similar to intraocular device 101 of FIGS. 6A-7, and thus will be described herein to the extent necessary to identify differences in construction and operation thereof. Throughout FIGS. 6A-11 of the present disclosure, like reference numerals (i.e., 101, 201, etc.) will be used to identify like elements.

The intraocular device 201 includes a plurality of flange groove parts 229 and wall groove parts 233. As shown in FIGS. 8A and 8B, the wall groove parts 233 may be arranged across the inner surface 222 of the vertical wall 220 in an asymmetrical pattern, and/or the flange groove parts 229 may be arranged across the proximal-facing second surface 219 of the annular flange 217 in an asymmetrical pattern, respectively.

In some embodiments, as shown in FIG. 8C, the device 201 further includes a stop member 237. One or more supports 239 extend proximally from the second surface 219 of the flange 217 to secure the stop member 237 in a position which is spaced proximally from the second surface 219 of the annular flange 217. The one or more supports 239 are located between neighboring spaced flange groove parts 229 to avoid blocking the flange groove parts 229 from allowing fluids to flow therethrough.

The stop member 237 is configured to limit and/or prevent distal movement of a fluid injection device beyond the stop member 237. In addition, the stop member 237 may be used to secure a fluid injection device to the intraocular device 201.

The stop member 237 includes an aperture 241 defined therethrough. The aperture 241 is configured to receive a luer connector (i.e., a luer nub or a luer lock) therein or therearound. The luer connector may be located on the distal end of the fluid injection device therein.

As further depicted in FIG. 8C, in some embodiments, the device 201 includes more than one internal channel 215, 235. For example, the device 201 includes a first internal channel 215 and a second internal channel 235. The first internal channel 215 is in fluid communication with the bore 225 and the plurality of flange groove parts 229. The first internal channel 215 is not in fluid communication with the second internal channel 235.

The second internal channel 235 extends distally from a patient-facing side of the stop member 237 and through the first internal channel 215. A distal end 236 of the second internal channel 235 may extend beyond a distal end of the first internal channel 215. The first internal channel 215 surrounds the second internal channel 235. The second internal channel 235 is in fluid communication with only the bore 225 via the aperture 241 defined through the stop member 237.

In still other embodiments, as further depicted in FIGS. 8A-8C, the device 201 includes a vertical wall 220 which extends generally perpendicular to the annular flange 217 forming a cylindrical vertical wall 220.

FIG. 9 illustrates the device 201 combined with a fluid injection device 250, such as a syringe barrel 252 including a distal luer connector 254 and free of a needle and/or cannula. The luer connector 254 may be a luer nub or a luer lock connector. As illustrated, the distal luer connector 254 is secured within the aperture 241 of the stop member 237 to put the fluid injection device 250 into fluid communication with the second internal channel 235 to deliver fluids F₁ into an eye (not shown) without a needle. Also, a distal end of the injection device 250 (and/or syringe barrel 252) is secured inside cylindrical vertical wall 220 (and/or bore 225) such that physiological fluids flow F₂ out of the eye (and/or the intraocular device 201) under pressure via the one or more grooves. The cylindrical vertical wall 220 may better secure the injection device 250 in the bore 225.

FIGS. 10A-10C depict various views of yet another intraocular device 301 as provided in yet another embodiment. Intraocular device 301 is similar to intraocular devices 101, 201, and thus will be described herein to the extent necessary to identify differences in construction and operation thereof.

In some embodiments, as shown in FIGS. 10A and 10C, the intraocular device 301 includes a non-linear and/or curved elongate tubular portion 307, which defines at least one inner channel 315 extending therethrough which is also non-linear and/or curved. The non-linear tubular structure 107 may define an outer perimeter which narrows distally along a length thereof.

In some embodiments, the device 301 includes an outer surface 323 of a vertical wall 320 which defines a non-cylindrical or non-conical configuration, and an inner surface 322 which defines a different configuration than the outer surface 323. For example, as shown, the outer surface 323 of the vertical wall 320 may define an octagonal shape while the inner surface 322 remains cylindrical or conical. It is envisioned that a cylindrical or conical inner surface 322 may be most useful as many fluid injection devices define a generally circular exterior, while non-circular outer surfaces 323 may be easier to handle. However, the inner and outer surfaces 322, 323, in any combination, may define any geometric shape or design, including triangular, rectangular, square, elliptical, pentagonal, circular, star-shaped, and the like.

As further depicted in FIGS. 10A-10C, the intraocular device 301 may also include one or more closed wall groove parts 333 which are not open to or in fluid communication with the bore 325. Rather, the closed wall groove parts 333 are defined within the thickness of the vertical wall 320 and include an opening 360 defined within the thickness of the vertical wall along a top side 321 of the vertical wall 320.

FIG. 10D schematically represents an intraocular device 301 which is designed to be separated into two or more pieces, at any time but particularly post-operatively. The separatable device 301 includes a means 365 for dividing the device 301 into separate pieces. For example, in some embodiments, the device 301 may be a one-piece device which includes a separation means such as perforations 365 spaced around a distal end of the vertical wall 320. Ideally, the perforations 365 are located between the radially spaced wall groove parts 333 so as not to affect the flow of fluids therethrough. In another example, in some embodiments, the device 301 may be made of 2 or more separate pieces which can be combined and separated along means 365 using any type of male/female connector (e.g., snap-fit connectors, friction-fit connectors, luer connectors, etc.).

As depicted, the vertical wall 320 is configured to be separated from the annular flange 317 post-operatively to remove the vertical wall 320. Removal of the wall 320 may be beneficial so as not to extend outwardly from the post-operative eye E. Removal of the wall 320 removes a need for additional bandaging to stabilize the device post-operatively. The vertical wall 320 will likely also interfere with a proper blinking process of the eyeball E. By removing the vertical wall 320 and leaving the elongate tubular portion 307 within the eye E with the annular flange 317 positioned along an outer surface of the eye E, the intraocular pressure can continue to be controlled without the need for additional support of the device 301 and/or without interfering with a proper blinking process of the post-surgical eyeball E.

In some embodiments, the annular flange 317 and/or the at least one internal channel 315 may further include one or more valves 369 as provided herein to monitor and/or control the outward flow of physiological fluids. Alternatively, or in combination, in some embodiments, the one or more valves 369 can be located in the closed wall groove parts.

The intraocular devices provided herein may be made from any biocompatible and/or sterilizable material. The intraocular devices provided herein may also be manufactured using any suitable process including molding, pressing, casting, and/or 3-D printing.

The present disclosure describes various intraocular devices including various different elements. The various elements are not only intended to be combined with the other elements of a given device as expressly described herein, but the various elements are also intended to be combined with any of the other various elements expressly described herein and/or incorporated into any of the other various intraocular devices provided.

1. A device for temporary use in cataract surgery and post-operative surgical care comprising:
an elongate tubular structure including at least one internal channel extending between a proximal end of the tubular structure and a distal end of the tubular structure, the proximal end of the tubular structure including an annular flange extending axially therefrom, the annular flange having a patient-facing first surface and an opposite proximally-facing second surface,
a vertical wall extending proximally from the annular flange, the vertical wall and the second surface of the annular flange defining a bore therein configured to receive a fluid injection apparatus, the bore being in fluid communication with a proximal end of the internal channel, and
one or more grooves in fluid communication with the proximal end of the internal channel to permit physiologic fluids to pass through the device to balance intraocular pressure, the one or more grooves extending along the annular flange and the vertical wall.

2. The device of claim 1, wherein the elongate tubular portion is generally linear.

3. The device of claim 1, wherein the elongate tubular portion is generally curved.

4. The device of claim 1, wherein the vertical wall comprises a thickness defined between an inner surface of the vertical wall and an outer surface of the vertical, the inner surface of the vertical wall generally defines an outer perimeter of the bore.

5. The device of claim 4, wherein the one or more grooves are a plurality of grooves spaced radially about the annular flange and the vertical wall.

6. The device of claim 5, wherein each groove of the plurality of grooves includes:
a flange groove part which extends radially along the second surface of the annular flange from the internal channel towards the vertical wall; and
a wall groove part which extends vertically along the vertical wall, the wall groove part in fluid communication with the flange groove part.

7. The device of claim 6, wherein the wall groove part is an open wall groove part defined within the inner surface of the vertical wall, the open wall groove part is also in fluid communication with the bore.

8. The device of claim 7, wherein the open wall groove parts are radially spaced around the outer perimeter of the bore intermittently expanding the outer perimeter of the bore to permit physiologic fluid to pass through the open wall groove parts while the fluid injection device is received within the bore.

9. The device of claim 8, wherein the open wall groove parts and vertical segments of the inner surface of the vertical wall alternate around the outer perimeter of the bore.

10. The device of claim 6, wherein the wall groove part is a closed wall groove part which is defined within the thickness of the vertical wall and not in fluid communication with the bore, wherein a proximal end of the closed wall groove part includes an opening defined within the thickness of the vertical wall along a top side of the vertical wall.

11. The device of claim 6, further comprising a stop member spaced proximally from the second surface of the annular flange, the stop member configured to prevent distal movement of a barrel of the fluid injection device beyond thereof.

12. The device of claim 11, wherein the stop member is spaced from the second surface of the annular flange by one or more supports extending therefrom and located between the radially spaced flange groove parts.

13. The device of claim 11, wherein the stop member includes an aperture defined therethrough, the aperture configured to receive a cannula located on a distal end of the barrel therethrough or a luer connector located on the distal end of the barrel therein.

14. The device of claim 11, wherein the at least one internal channel includes:
a first internal channel in fluid communication with the bore and the plurality of flange groove parts; and
a second internal channel extending distally from the stop member through the first internal channel, the second internal channel in fluid communication with the bore via the aperture defined within the stop member.

15. The device of claim 14, wherein a distal end of the second internal channel extends beyond a distal end of the first internal channel.

16. The device of claim 1, wherein the vertical wall is configured to be separated from the annular flange post-surgery.

17. The device of claim 4, wherein an outer perimeter of the vertical wall or the outer perimeter of the bore is larger than an outer perimeter of the at least one internal channel.

18. The device of claim 4, further comprising one or more raised ridges positioned on the outer surface of the vertical wall for handling of the device.

19. The device of claim 1, wherein a distal end of the elongate tubular portion includes a forked end including a plurality of spaced tines for penetrating the eyeball.

20. The device of claim 1, wherein a length of the elongate tubular portion is generally equal to a length of both the annular flange and vertical wall.
